(19) United States
(12) Patent Application Publication
(10) Pub. No.: US 2008/0102141 A1
(43) Pub. Date: May 1, 2008

(54) Use as a means to improve fertility of a phytotherapeutic composition and method for improving and/or promoting fertility health

(76) Inventors: Maria Antoinette Brandes, Norbury (GB); Jacobus Marinus Brandes, Norbury (GB)

Correspondence Address: LAW OFFICES OF ERIC KARICH, 2807 ST. MARK DR., MANSFIELD, TX, 76063, US

(21) Appl. No.: 11/589,496

(22) Filed: Oct. 30, 2006

(51) Int. Cl. A61K 36/254; A61K 36/28; A61P 15/08

(52) U.S. Cl. 424/728; 424/737

(57) ABSTRACT

Use as a means to improve fertility of a phytotherapeutic composition and method for improving and/or promoting fertility health. The phytotherapeutic composition comprising at least 6 volume % Allium Sativum extract, 17 volume % Eleutherococcus Senticosus extract, 11 volume % Juglans Regia extract, 17 volume % Echinacea Purpurea extract, 17 volume % Viola Tricolor extract, 22 volume % Uncaria Tomentosa extract, and 11 volume % Carduus Marianus extract is used to improve fertility of mammals.

### BACKGROUND OF THE INVENTION

1. Field of the Invention

The invention lies in the field of means and methods to improve/promote fertility of mammals and humans both in male and female.

2. Prior Art

Many means are known to improve/promote fertility of mammals and humans. For example from US 2005/0,287,228 a method of promoting fertility health in women comprising taking a dietary supplement of L-arginine, magnesium, green tea, vitamin E, Vitex agnus-castus, iron, zinc, selenium, vitamins B6 and B12 and folic acid, and a supplement for promoting women's fertility comprising an herb for enhancing hormone balance, increasing progesterone release and ovulation frequency and an amino acid for stimulating reproductive organs by improving circulation is known.

### SUMMARY OF THE INVENTION

An objective of the invention is to provide a means to improve fertility of mammals and humans both in male and female. In the present invention this objective is realised by using a phytotherapeutic composition comprising extracts of Allium sativum, Juglans regia, Echinacea purpurea and Viola tricolor.

It is noted that from the international application published under No. WO 01/034170 a phytotherapeutic composition is known to improve the condition of the immune system of a human or animal. It has been found that this phytotherapeutic composition has also a positive side effect on fertility of mammals and humans both male and female. In female, egg production went up as abnormalities in cycles disappeared and in male animals semen density, sperm cell count, morphology and motility all showed positive improvements. Embodiments of the use of this composition are described in the claims.

The active ingredients in herbs are often obtained by extraction from the herbs. The extracts are often administered dissolved in or mixed with water via oral ingestion.

The invention further relates to a method for improving and/or promoting fertility health in mammals and humans comprising taking a phytotherapeutic composition comprising extracts of Allium Sativum, Juglans Regia, Echinacea Purpurea and Viola Tricolor. Embodiments of this method are described in the claims

The invention also relates to a phytotherapeutic composition for improving and/or promoting fertility health in mammals and humans comprising 5-7 volume % Allium Sativum extract, 16-18 volume % Eleutherococcus Senticosus extract, 10-12 volume % Juglans Regia extract, 16-18 volume % Echinacea Purpurea extract, 16-18 volume % Viola Tricolor extract, 21-23 volume % Uncaria Tomentosa extract, and 10-12 volume % Carduus Marianus extract.

Preferably the phytotherapeutic composition according to the invention comprises 5.55 volume % Allium Sativum extract, 16.67 volume % Eleutherococcus Senticosus extract, 11.11 volume % Juglans Regia extract, 16.67 volume % Echinacea Purpurea extract, 16.67 volume % Viola Tricolor extract, 22.22 volume % Uncaria Tomentosa extract, and 11.11 volume % Carduus Marianus extract.

### DETAILED DESCRIPTION OF THE INVENTION

In one embodiment, a phytotherapeutic composition comprises a combination of extracts of the herbs Allium Sativum or garlic, Juglans Regia or walnut, Echinacea Purpurea or purple coneflower, Viola Tricolor or Violet, Carduus Marianus or milk thistle, Uncaria Tomentosa or Cat's claw and Eleutherococcus Senticosus or Siberian Ginseng, in the ratio of about 1:2:3:3:2:4:3.

The phytotherapeutic composition preferably comprises at least about 5.55 volume % Allium Sativum extract, 16.67 volume % Eleutherococcus Senticosus extract, 11.11 volume % Juglans Regia extract, 16.67 volume % Echinacea Purpurea extract, 16.67 volume % Viola Tricolor extract, 22.22 volume % Uncaria Tomentosa extract, and 11.11 volume % Carduus Marianus extract. This composition has proven to have a very positive effect on fertility improvement in female and semen improvement in male.

The extracts are obtained in a manner known to experts by extracting the herbs, for example on a water or alcohol basis. Preferably the phytotherapeutic composition is comprised of alcohol extracts of the above-mentioned herbs with an alcohol content of approximately 40%.

In addition to the above-mentioned herbs, the phytotherapeutic composition can also contain other components, such as for example scents and flavours if the medication is administered orally.

Although in the above the invention is explained on the basis of the description of one embodiment, it should be noted that the invention is in no way limited to this embodiment. The invention also extends to all embodiments deviating from this embodiment within the context defined by the claims. For example extraction can also take place on a watery acid basis.

1. Use as a means to improve fertility of mammals of a phytotherapeutic composition comprising extracts of Allium Sativum, Juglans Regia, Echinacea Purpurea and Viola Tricolor.

2. Use according to claim 1, wherein the ratio of the quantity of the extracts Allium Sativum, Juglans Regia, Echinacea Purpurea and Viola Tricolor is approximately 1:2:3:3.

3. Use according to claim 1, wherein the phytotherapeutic composition further comprises an extract of Carduus Marianus.

4. Use according to claim 3, wherein the ratio of the quantity of the extracts Allium Sativum, Juglans Regia, Echinacea Purpurea, Viola Tricolor, and Carduus Marianus is approximately 1:2:3:3:2.

5. Use according to claim 1, wherein the phytotherapeutic composition further comprises an extract of Uncaria Tomentosa.

6. Use according to claim 3, wherein the phytotherapeutic composition further comprises an extract of Uncaria Tomentosa.

7. Use according to claim 6, wherein the ratio of the quantity of the extracts Allium Sativum, Juglans Regia, Echinacea Purpurea, Viola Tricolor, Carduus Marianus and Uncaria Tomentosa is approximately 1:2:3:3:2:4.

8. Use according to claim 1, wherein the phytotherapeutic composition further comprises an extract of Eleutherococcus Senticosus.

9. Use according to claim 6, wherein the phytotherapeutic composition further comprises an extract of Eleutherococcus Senticosus.

10. Use according to claim 9, wherein the ratio of the quantity of the extracts Allium Sativum, Juglans Regia, Echinacea Purpurea, Viola Tricolor, Carduus Marianus, Uncaria Tomentosa and Eleutherococcus Senticosus is approximately 1:2:3:3:2:4:3.

11. Use according to claim 10, wherein the phytotherapeutic composition comprises 1-20 volume % Allium Sativum extract, 5-25 volume % Eleutherococcus Senticosus extract, 1-20 volume % Juglans Regia extract, 5-25 volume % Echinacea Purpurea extract, 5-25 volume % Viola Tricolor extract, 10-30 volume % Uncaria Tomentosa extract, and 1-20 volume % Carduus Marianus extract.

12. Use according to claim 10, wherein the phytotherapeutic composition comprises 5-7 volume % Allium Sativum extract, 16-18 volume % Eleutherococcus Senticosus extract, 10-12 volume % Juglans Regia extract, 16-18 volume % Echinacea Purpurea extract, 16-18 volume % Viola Tricolor extract, 21-23 volume % Uncaria Tomentosa extract, and 10-12 volume % Carduus Marianus extract.

13. A method for improving and/or promoting fertility health in mammals comprising taking a phytotherapeutic composition comprising extracts of Allium Sativum, Juglans Regia, Echinacea Purpurea and Viola Tricolor.

14. The method of claim 13, wherein the ratio of the quantity of the extracts Allium Sativum, Juglans Regia, Echinacea Purpurea and Viola Tricolor is approximately 1:2:3:3.

15. The method of claim 13, wherein the phytotherapeutic composition further comprises an extract of Carduus Marianus.

16. The method of claim 15, wherein the ratio of the quantity of the extracts Allium Sativum, Juglans Regia, Echinacea Purpurea, Viola Tricolor, and Carduus Marianus is approximately 1:2:3:3:2.

17. The method of claim 13, wherein the phytotherapeutic composition further comprises an extract of Uncaria Tomentosa.

18. The method of claim 15, wherein the phytotherapeutic composition further comprises an extract of Uncaria Tomentosa.

19. The method of claim 18, wherein the ratio of the quantity of the extracts Allium Sativum, Juglans Regia, Echinacea Purpurea, Viola Tricolor, Carduus Marianus and Uncaria Tomentosa is approximately 1:2:3:3:2:4.

20. The method of claim 13, wherein the phytotherapeutic composition further comprises an extract of Eleutherococcus Senticosus.

21. The method of claim 18, wherein the phytotherapeutic composition further comprises an extract of Eleutherococcus Senticosus.

22. The method of claim 21, wherein the ratio of the quantity of the extracts Allium Sativum, Juglans Regia, Echinacea Purpurea, Viola Tricolor, Carduus Marianus, Uncaria Tomentosa and Eleutherococcus Senticosus is approximately 1:2:3:3:2:4:3.

23. The method of claim 22, wherein the phytotherapeutic composition comprises 1-20 volume % Allium Sativum extract, 5-25 volume % Eleutherococcus Senticosus extract, 1-20 volume % Juglans Regia extract, 5-25 volume % Echinacea Purpurea extract, 5-25 volume % Viola Tricolor extract, 10-30 volume % Uncaria Tomentosa extract, and 1-20 volume % Carduus Marianus extract.

24. The method of claim 23, wherein the phytotherapeutic composition comprises 5-7 volume % Allium Sativum extract, 16-18 volume % Eleutherococcus Senticosus extract, 10-12 volume % Juglans Regia extract, 16-18 volume % Echinacea Purpurea extract, 16-18 volume % Viola Tricolor extract, 21-23 volume % Uncaria Tomentosa extract, and 10-12 volume % Carduus Marianus extract.

25. A phytotherapeutic composition comprising 5-7 volume % Allium Sativum extract, 16-18 volume % Eleutherococcus Senticosus extract, 10-12 volume % Juglans Regia extract, 16-18 volume % Echinacea Purpurea extract, 16-18 volume % Viola Tricolor extract, 21-23 volume % Uncaria Tomentosa extract, and 10-12 volume % Carduus Marianus extract.

26. The phytotherapeutic composition of claim 25 comprising 5.55 volume % Allium Sativum extract, 16.67 volume % Eleutherococcus Senticosus extract, 11.11 volume % Juglans Regia extract, 16.67 volume % Echinacea Purpurea extract, 16.67 volume % Viola Tricolor extract, 22.22 volume % Uncaria Tomentosa extract, and 11.11 volume % Carduus Marianus extract.
